(19) United States
(12) Patent Application Publication
(10) Pub. No.: US 2025/0082351 A1
(43) Pub. Date: Mar. 13, 2025

(54) DEVICE FOR THE ENDOSCOPIC REMOVAL OF STONES OR CONCREMENTS FROM THE BILE AND PANCREATIC DUCT AND METHOD FOR CONVERTING A DEVICE ARRANGED IN A WORKING CHANNEL OF A MEDICAL ENDOSCOPE

(71) Applicant: FUJIFILM CORPORATION, Tokyo (JP)

(72) Inventors: Danijel KELM, Burgebrach (DE); Thassilo AMON, Strullendorf (DE); Igor GORTE, Adelsdorf (DE)

(21) Appl. No.: 18/883,071

(22) Filed: Sep. 12, 2024

(30) Foreign Application Priority Data
Sep. 13, 2023 (EP) 23 197 140.9

(51) Int. Cl. A61B 17/221; A61B 17/00; A61B 17/22

(52) U.S. Cl. CPC A61B 17/221; A61B 17/2202; A61B 2017/00818; A61B 2017/2215; A61B 2017/2217

(57) ABSTRACT

A device for the endoscopic removal of stones or concrements from the bile and pancreatic duct has a control wire guided in a tube, at the proximal end of which a control element for the axial displacement of the control wire within the tube and at the distal end of which a catch basket made of basket wires are fastened. Axial displacement of the control wire by the control element leads to a loop-like expansion or contraction of the catch basket. The control wire is separable from the control element and is couplable for lithotripsy of the stone picked up by the catch basket to an actuating element, with which a sufficient force is producible on the catch basket.

### CROSS-REFERENCE TO RELATED APPLICATIONS

The present application claims priority of EP 23 197 140.9, filed Sep. 13, 2023, the priority of this application is hereby claimed, and this application is incorporated herein by reference.

### BACKGROUND OF THE INVENTION

The invention relates to a device for the endoscopic removal of stones or concrements from the bile and pancreatic duct, having a control wire which is guided in a tube and at the proximal end of which a control element for the axial displacement of the control wire within the tube and at the distal end of which a catch basket consisting of basket wires are fastened, wherein an axial displacement of the control wire by means of the control element leads to a loop-like expansion or contraction of the catch basket.

Furthermore, the invention also relates to a method for converting a device arranged in a working channel of a medical endoscope for use for lithotripsy and removal of a stone captured by a catch basket, being performable after endoscopic retrograde cholangiopancreatography, wherein the device comprises a tube, a control wire guided therein, a catch basket connected distally to the control wire for receiving a stone and a control element engaging the tube and the control wire, and wherein the catch basket is expanded or contracted in a loop-like manner by an axial displacement of the control wire by means of the control element.

Endoscopic retrograde cholangiopancreatography (ERCP) is an endoscopic procedure used to visualize and examine a patient's bile and pancreatic duct. A medical endoscope is inserted into the patient's esophagus via the pharynx, whereby one distal end of the endoscope is fitted with a camera designed as a side optic and passes through the stomach into the duodenum. For further examination and possible treatment of the patient's bile and pancreatic ducts, various endoscopic devices equipped with distal instruments are guided via the working channel of the medical endoscope into the area of the bile and pancreatic ducts to be treated. These can be used to open narrowed or blocked ducts, remove gallstones and/or break them up as part of a lithotripsy, perform a biopsy and possibly surgically remove tumors in the area of the ducts and insert stents into the ducts. Using endoscopic retrograde cholangiopancreatography (ERCP) after papillotomy, a gallstone is usually extracted using a catch basket. The catch basket is also suitable for the safe removal of stone fragments from the bile duct.

A device for the endoscopic removal of stones or concrements from the bile and pancreatic duct of the type mentioned in the generic term of claim 1 is known from DE 10 2009 031 209 A1. This has a cable guided in a tube, at the proximal end of which is arranged a control element for axial displacement of the cable within the tube and at the distal end of which is arranged a catch basket formed from at least four elastic basket wires. When the cable is axially displaced by means of the control element, the catch basket is pulled together like a loop or widened, as the catch basket is pulled towards a distal end of the tube and partially into its lumen. The capture basket is used to capture a gallstone that has been released from the papilla and is moved rotationally to capture it, while at the same time narrowing. The gallstone trapped between the basket wires is fixed in the catcher basket so that it can then be removed from the bile and pancreatic duct.

Significant problems always arise when the dimensions of the stone do not allow it to be pulled through body orifices, such as the bile duct, for its removal. Forcible removal of the stone would then lead to considerable injury to the patient being treated. To counter this problem, the only options are to design the catch basket in such a way that via the catch basket a force is exerted on the stone for its lithotripsy or that the catch basket can be actuated via the endoscopic device to release the stone so that the device can be removed from the bile duct after the catch basket has been separated from the stone.

Furthermore, DE 32 16 178 A1 discloses a device for mechanical lithotripsy and for removing stones that have formed in the bile duct. This device consists of an extractor and can be combined with a device for lithotripsy if required. The extractor has a control device in the form of a handle connected to a push rod and a sleeve holding the push rod. The push rod is telescopically guided in the sleeve and its end facing away from the handle is fixed to the pull cable leading to the stone catch basket. Consequently, if the push rod is displaced axially in the sleeve, this leads, depending on the direction of movement, to an opening or closing of the stone catch basket, so that a stone present in the bile duct, for example, can be picked up by the stone catch basket and removed from the patient's body by moving the entire device in a proximal direction.

According to DE 32 16 178 A1 and in addition to the extractor, a separate lithotriptor is provided, which is fixed to the actuating element as required. This is a tensioning element with brackets running radially to it, which, after the lithotriptor has been attached, engage on the one hand on the handle of the first actuating element and on the other hand on a head piece connected to the sleeve. If it is determined that the stone has dimensions that prevent it from being easily removed, the lithotriptor attached to the actuating device exerts such a strong pulling force on the cable that the stone in the stone catcher basket breaks up into several small particles. These particles can then be removed with the stone catcher basket.

### SUMMARY OF THE INVENTION

It is the task of the present invention to improve a device for the endoscopic removal of stones or concrements from the bile and pancreatic duct in such a way that more favorable possibilities for their handling with regard to a necessary lithotripsy arise.

This problem is solved by a device for the endoscopic removal of stones or concrements from the bile and pancreatic duct, which has a control wire guided in a tube. A control element for axial displacement of the control wire is attached to a proximal end of the control wire running inside the tube, while a catch basket consisting of basket wires is attached to the distal end of the control wire. Axial displacement of the control wire by means of the control element leads to a loop-like expansion or contraction of the catch basket. The control element can therefore be used to carry out precise movements in order to capture the stone and enclose it securely for removal.

According to the invention, the control wire should be separable from the control element and should be couplable for lithotripsy of the stone received by the catch basket to an actuating element, with which a sufficient force can be generated on the catch basket. If, as already explained, the stone picked up by the catch basket has dimensions that prevent its removal through the bile duct, lithotripsy of the stone is required. This can be carried out by exerting such a force on the catch basket holding the stone, which is partially drawn into the lumen of the tube via the control wire and thus narrows, that the stone, crushed by mechanical lithotripsy, disintegrates into a large number of stone fragments remaining in the catch basket.

The control element acting on the control wire and being preferably provided with a thumb ring and two finger rings for one-handed operation as a slider cannot apply the necessary tensile force. In the solution according to the invention, the control element is replaced by an actuating element suitable for subsequent lithotripsy. For this purpose, the control element is separated from the control wire, which can then be coupled to the actuating element.

In contrast, according to the generic DE 10 2009 031 209 A1, the endoscopic device is intended to remove a gallstone by means of a catch basket; however, no mechanical lithotripsy is carried out with this, as the control element is only intended to carry out movements and to expand and contract the catch basket. Therefore, the control element provided on the device cannot be used to generate the tensile force required for mechanical lithotripsy on the catch basket.

According to the further prior art DE 32 16 178 A1 a separate lithotriptor is provided, but this must be fixed to the control element if necessary so that it can be used to achieve a sufficient tensile force on the control wire. However, this lithotriptor is not suitable for combination with every type of control element. In particular, this lithotriptor cannot be combined with a conventional control element that can be operated with one hand and has a thumb ring and two finger rings.

The actuating element should be designed and dimensioned in such a way that it can be used to apply a tensile force of up to 500 N for lithotripsy of the stone picked up by the catch basket. This high tensile force, which generates sufficient force on the catch basket to crush the stone, should neither lead to plastic deformation of the actuating element nor cause it to become brittle. A tensile force of 200 N is required to crush a gallstone with a diameter of approx. 1 cm.

In a further embodiment of the invention, a traction means extends from the actuating element and is couplable to the control wire via a form-fitting connection. Consequently, the control wire, which is separated from the control element, is then connected to the traction means in a form-fit manner. The interlocking of the connecting partners of these two components, the traction means and the control wire, results in form-fitting connection that acts in the direction of the tensile force.

In a further embodiment of the invention, the form-fitting connection should be designed as a hook-eyelet-connection. In this case, the hook preferably formed on the control wire is hooked into a distal eyelet of the pulling means. The high tensile force generated by the actuating element is thus transmitted using simple means.

As further intended, the control wire can be provided with a kink at its proximal end section after its separation from the control element, whereby the kinked proximal end section runs essentially parallel to the control wire. The kink acts as a hook for the form-fitting connection with the eyelet, and means can be provided to prevent the hook from releasing the eyelet under the high tensile force.

Furthermore, means for fixing the kink should interact with the proximal end section. These means prevent the kink from bending open under the high tensile force transmitted to it by the eyelet. Preferably, the means are created by drawing the proximal end section of the control wire suspended in the eyelet of the pulling means into a lumen of a spiral tube made of metal, thus reliably preventing the kink from bending open. A diameter of the lumen formed in the spiral hose is dimensioned in such a way that, on the one hand, the proximal section can be drawn into it and moved within it and, on the other hand, a radially acting tension acts on the kinked proximal end section, which receives the eyelet, so that high tensile forces can be transmitted with this connection.

It is also provided that the actuating element has a shaft extending in the same longitudinal direction as the traction means with a tensioning device extending transversely thereto and rotatably mounted thereon, into which a proximal end of the traction means can be suspended. The proximal end of the traction means, which is designed as a wire or cable, is wound onto the tensioning device, which is designed like a spool, by a rotary movement manually generated by the person carrying out the treatment on the tensioning device.

In a further embodiment of the device according to the invention, the kink in the proximal end section of the control wire is to be producible by means of a tool which has two tool parts which perform an angular movement relative to one another. According to a first embodiment, the tool can have two legs connected to each other via a hinge, whereby the control wire can be fixed to one of the legs. Preferably, the legs are designed as flat plastic parts connected to each other at one of their edges via the hinge.

According to a second embodiment, a tool can also be provided with an annular base part having holding grips with a circular disk rotatably guided therein. In this case, the proximal end section is inserted radially via an insertion opening of the base part both into the base part and into an adjoining driver of the disk. If the disk is now rotated relative to the base part, this leads to a hook-like deformation of the proximal end section.

In addition, the tool with legs connected via the hinge may have aligned grooves for receiving the control wire and a clamp for fixing the control wire may be provided on at least one leg.

Furthermore, the problem underlying the invention is also solved in the context of a method for converting a device which is arranged in a working channel of a medical endoscope and is used after endoscopic retrograde cholangiopancreatography, wherein the device is used for lithotripsy and removal of a stone captured by a catch basket and has a tube, a control wire guided in the tube, a catch basket connected distally to the control wire for receiving a stone and a control element engaging on the tube and on the control wire, and wherein the catch basket is widened or contracted in a loop-like manner by an axial displacement of the control wire by means of the control element.

According to the invention, the following process steps are provided:
Cutting of the control wire in its proximal section, in which it is guided in a guide shaft of the control element;
Kinking of the control wire in a proximal end section to form a hook-shaped configuration;
Suspension of the proximal hook-shaped end section of the control wire in an eyelet of a pulling means of an actuating element used for lithotripsy;
Movement of the hook-shaped end of the control wire and the eyelet of the pulling means in the longitudinal direction into a spiral hose made of metal by tensioning the actuating element, and
further tensioning of the actuating element to generate a force on the catch basket for crushing the stone picked up by the latter.

Furthermore, as part of the method, after the end section provided with the kink has been hooked in, the end section provided with the kink can first be manually pulled in a proximal direction until the eyelet is in contact with the distal end of the spiral hose. The proximal end of the pulling means is then connected to a tensioning device arranged on the actuating element and the pulling means is then tensioned via the tensioning device, which is designed as a winding spool, until the spiral hose is pushed over the eyelet. The tensile force transmitted to the control wire is then increased by a further actuation of the tensioning device until the catch basket is moved to the distal end of the tube. Finally, the stone picked up by the catch basket is crushed by a further increase in the tensile force.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

FIG. 1 a side view of a control element of a device for the endoscopic removal of stones or concrements from the bile and pancreatic duct, shown in perspective, in which only a section of a tube is visible,

FIG. 2 side view of a distal end section of the control wire with a catch basket attached to it,

FIG. 3 a separation of the control element from the control wire by means of a side cutter after it has been determined during a medical endoscopic procedure on the patient that a lithotripsy must be performed with considerable force on the stone picked up by the catch basket,

FIG. 4 a proximal end of the control wire inserted into a tool used to bend its end portion into a hook,

FIG. 5 the proximal end section of the control wire bent into a hook,

FIG. 6 a pulling means arranged in a metallic spiral hose and forming an eyelet at its distal end and being coupled to the hook via this eyelet,

FIG. 7 an actuating element used for lithotripsy, to which a proximal end of the traction means is attached,

FIG. 8 the tool used according to FIG. 4 for bending the proximal end portion of the control wire in its state in which the control wire can be inserted into it,

FIG. 9 the tool shown in FIG. 8 in its closed state, in which the bending of the control wire is effected,

FIG. 10 an alternative embodiment of a tool for bending the control wire and

FIG. 11 the actuating element used as shown in FIG. 7.

### DETAILED DESCRIPTION OF THE INVENTION

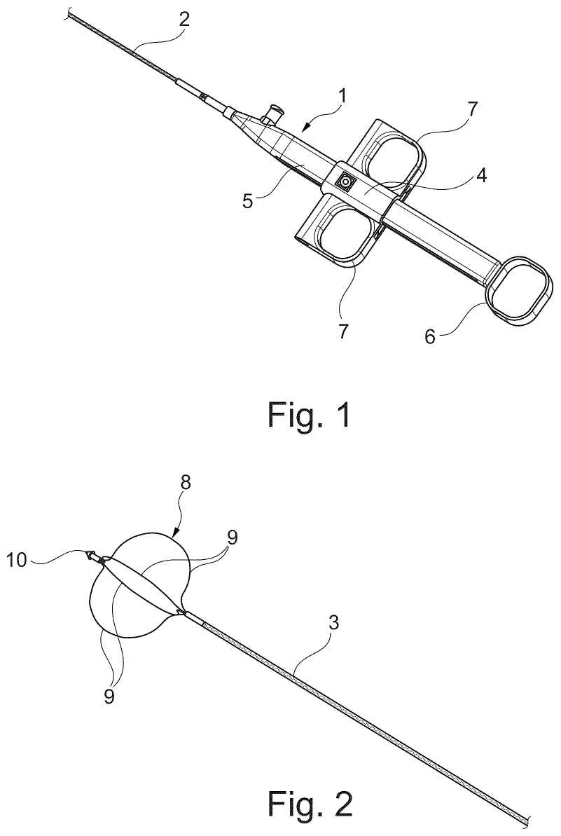

In FIG. 1, 1 indicates a control element, from which a tube 2 and a control wire 3 extend, which runs in a lumen of the tube 2 that is not shown in detail and can be seen in FIG. 2. The control element 1 has a slider 4, which is connected to the proximal end of the control wire 3 and is guided in a longitudinal slot of a guide shaft 5, which is not shown in detail. This guide shaft 5 is in turn firmly connected to the tube 2, so that the control wire 3 can be moved back and forth within the tube 2 by means of a relative movement of the slider 4 with respect to the guide shaft 5. To carry out these movements, the guide shaft 5 is provided with a thumb ring 6, while the slider has two finger rings 7.

FIG. 2 shows a distal end section of the control wire 3 without the tube 2 surrounding it, whereby a catch basket 8 is fixed at the distal end. Basket wires 9 of this catch basket 8, which is open in the illustration, are bundled at their respective ends, on the one hand at the end of the control wire 3 and on the other hand in a cartridge-shaped guide element 10 forming the distal end of the catch basket 8.

The tube 2 together with the control wire 3 and the catch basket 8 is preferably inserted into the working channel of a medical endoscope that extends via the pharynx, esophagus and stomach of a patient into the area of the bile and pancreatic duct of a patient and that is not shown. Here, the catch basket 8 is initially in its closed position, in which it is completely contained by the tube 2. Via a guide wire, which can preferably be inserted into a second lumen of the tube 2, is inserted into the body opening and extends into the area of a stone present under endoscopic or radiological observation, the tube 2, which accommodates the guide wire 3 and the catch basket 8, is then guided.

The catch basket 8 is then actuated via the control element 1 to its open state shown in FIG. 2, whereby this is due to the inherent tension of the catch basket 8. In this open position, the open catch basket 8 positioned by control element 1 can pick up the stone. Preferably, the catch basket 8 performs a rotary movement during the pick-up of the stone and its subsequent closing movement caused by the control element 1, during which the catch basket 8 is supported at the distal end of the tube 2, which makes it easier to pick up the stone. During a subsequent pulling movement of the control wire 3, which is moved relative to the tube 2, the catch basket 8 contracts tightly around the stone and encloses it so that it can be safely removed, braced by the control wire 3 and the tube 2 in the catch basket 8, passing through the corresponding body openings.

Significant problems arise, however, if the stone is so large that it cannot be pulled through body orifices such as the bile duct. Forcible removal of the stone would lead to considerable injury to the patient being treated. However, with the control element 1, which is primarily suitable for localizing and picking up the stone, it is also not possible to transmit sufficient tractive force via the control wire 3 to the catch basket 8 to break up the stone. Therefore, the only remaining option is to open the catch basket 8 via a release device provided on it in order to release the stone. However, the previous processes must then be repeated in order to pick up the stone with a basket of a separate lithotriptor.

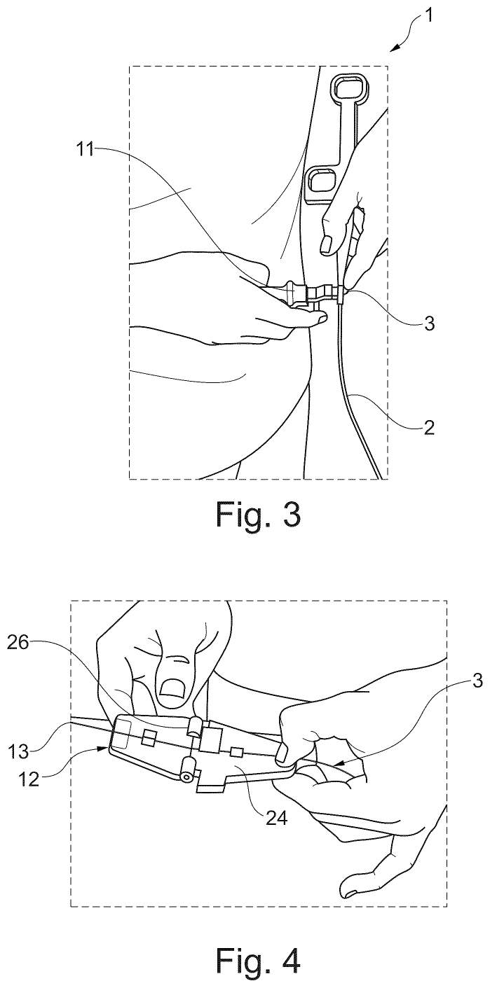

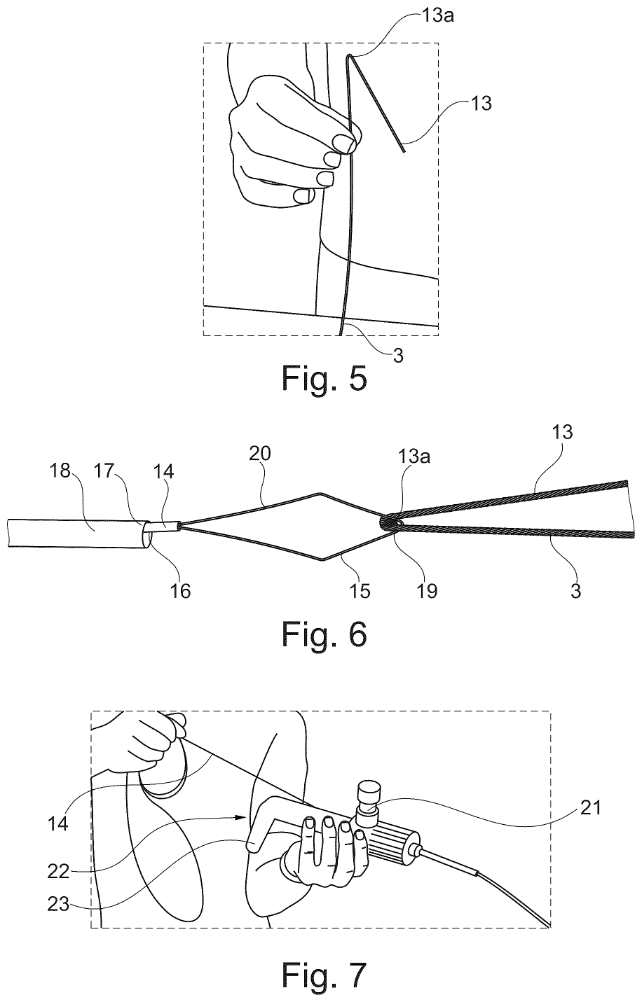

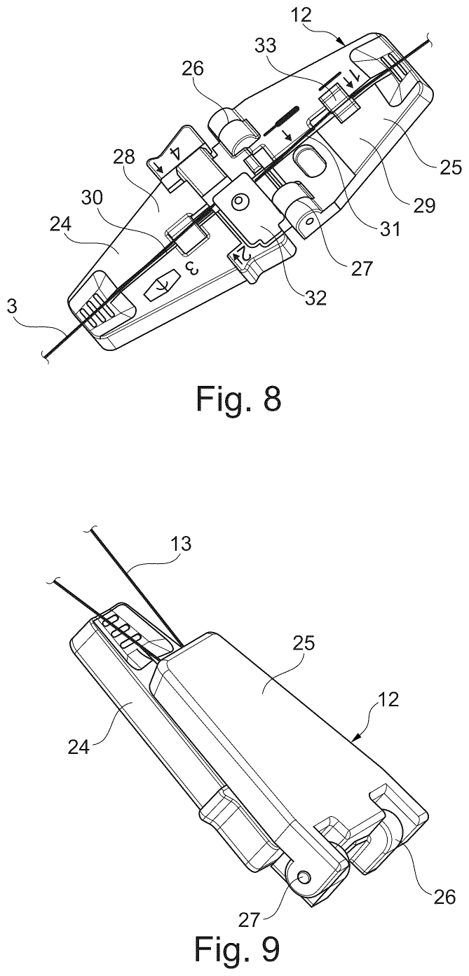

According to the invention, if a lithotripsy of the stone that is too large for normal removal is required, as can be seen in FIG. 3, the tube 2 and the control wire 3 are separated proximally from the control element 1 by means of a side cutter 11. The control wire 3 is separated in such a way that a proximal end section 13 of it still protrudes axially beyond the proximal end of the tube 2. As can be seen from FIG. 4, this protruding end section 13 of the control wire 3 is inserted into a tool 12 and, as will be explained in more detail below in connection with FIG. 8, is fixed in this tool 12. A proximal end section 13, which is shown in FIG. 5, is bent over in the shape of a hook with the tool 12, whereby a kink 13a is formed in the proximal end section 13.

As shown in FIG. 6, the hook-shaped bent end section 13 is hooked into an eyelet 15 provided distally on a traction means 14 designed as a wire or cable, which has a diamond-shaped outer contour. The traction means 14 runs in a lumen 16 of a spiral tube 17 made of metal, which is arranged in a tube 18. The diamond-shaped outer contour of the eyelet 15 ensures that the hook-shaped end section of the control wire 3 suspended in it is caught in a distal acute-angled region 19 of the eyelet 15 and eyelet wires 20 are drawn together radially when the eyelet 15 is pulled into the lumen 16.

According to FIG. 7, the traction means 14 is pulled manually in a proximal direction and then hooked into a tensioning device 21 of an actuating element 22. The tensioning device 21 extends at right angles to a handle part 23 of the actuating element 22 and is rotatably mounted on the latter. If the tensioning device 21 is thus manually actuated in such a way that it performs a rotary movement relative to the handle part 23, this causes the traction means 14 to be wound up on a correspondingly designed section of the tensioning device 21.

As a result, the eyelet 15 protruding from the distal end of the tube 18 as shown in FIG. 6 is pulled into the lumen 16 of the spiral tube 17 together with the proximal end section 13 of the control wire 3 hooked into it. As a result, an increasing tensile force occurs both in the pulling means 14 and in the control wire 3 coupled to it, which is transmitted to the catch basket 8 for lithotripsy of the corresponding stone.

The other FIGS. 8 and 9 show different perspectives of the tool 12 used to bend the control wire 3, the function of which has already been explained in connection with FIG. 4. The tool 12 consists of two legs 24 and 25 that are pivotably guided relative to each other and are connected to each other via hinges 26 and 27. In FIG. 8, the tool 12 is in an open state, whereby surfaces 28 and 29 of the two legs 24 and 25 are shown, which are moved towards each other when a control wire 3 is bent, guided on the hinges 26 and 27.

There are aligned grooves 30 and 31 in the surfaces 28 and 29 of the legs 24 and 25, into which the control wire 3 is inserted before the bending process. The control wire 3 is then held in its position in the two grooves 30 and 31 by a spring plate 32 provided on the leg 24 and a clamp 33 arranged on the leg 25. The tool 12 is then folded up, as shown in FIG. 9, so that the proximal end section 13 of the control wire 3 received by it is deformed into the hook-shaped configuration shown in FIG. 5.

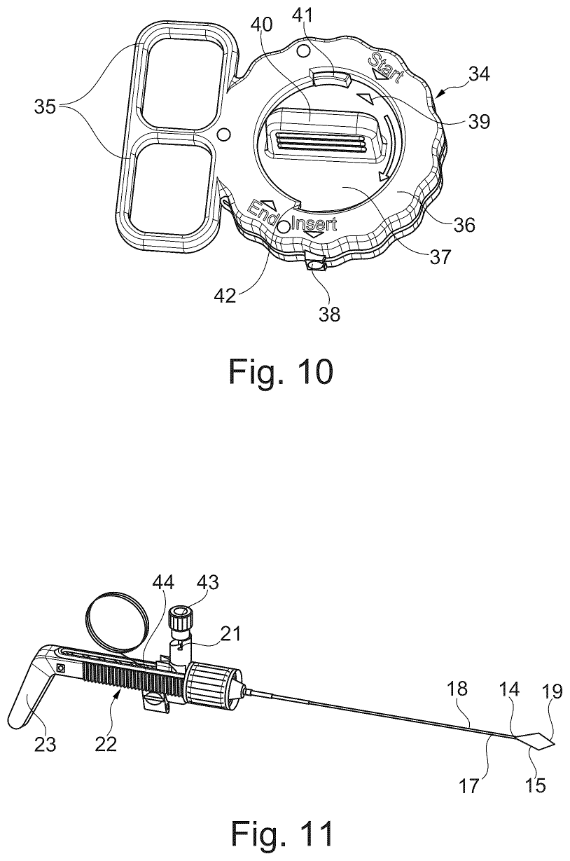

FIG. 10 shows an alternative embodiment of a tool 34, which consists of an annular base part 36 provided with handles 35 and a circular disk 37 rotatably guided therein. In this case, the proximal end section 13 shown in FIG. 4 is inserted via an insertion opening 38 of the base part 36 both into the base part 36 and into an adjoining channel of the disk 37, which is not shown in detail.

This takes place in a position of the disk 37 in relation to the base part 36, in which an arrow 39 points to a “Start” marking. An actuating lever 40 on the disk 37 is then used to turn it clockwise from this position to an end position defined by end stops 41 and 42. This bends the part of the inserted control wire 3 that is guided between the base part 36 and the disk 37.

Finally, FIG. 11 shows the actuating element 22 already explained in connection with FIG. 7, in the tensioning device 21 of which the tensioning means 14 is suspended. When the handle part 43, which is formed on the tensioning device 21 and extends at right angles to a shaft 44 and is provided with a knurling, is rotated, this leads to a displacement of the traction means 14 within the tube 18 in the proximal direction, as a result of which the eyelet 15 projecting beyond the distal end of the tube is drawn into the spiral tube 17 arranged in the tube 18.

While specific embodiments of the invention have been shown and described in detail to illustrate the inventive principles, it will be understood that the invention may be embodied otherwise without departing from such principles.

We claim:

1. A device for the endoscopic removal of stones or concrements from the bile and pancreatic duct, having a control wire which is guided in a tube and at the proximal end of which a control element for the axial displacement of the control wire within the tube and at the distal end of which a catch basket consisting of basket wires are fastened, wherein an axial displacement of the control wire by means of the control element leads to a loop-like expansion or contraction of the catch basket, wherein the control wire is separable from the control element and is couplable for lithotripsy of the stone received by the catch basket to an actuating element, with which a sufficient force is producible on the catch basket.

2. The device according to claim 1, wherein a tractive force of up to 500 N is applicable with the actuating element in order to shatter the stone.

3. The device according to claim 1, wherein a traction means extends from the actuating element and is couplable to the control wire via a form-fitting connection.

4. The device according to claim 3, wherein the form-fitting connection is designed as a hook-eyelet-connection.

5. The device according to claim 4, wherein the control wire is provided with a kink at its proximal end section after its separation from the control element, the kinked proximal end section extending at an acute angle or parallel to the control wire.

6. The device according to claim 5, wherein means interact with the proximal end section for fixation of the kink.

7. The device according to claim 5, wherein the proximal end section of the control wire suspended in the eyelet of the traction means is retracted together with the eyelet into a lumen of a spiral tube made of metal and serves as a means for fixation of the kink.

8. The device according to claim 3, wherein the actuating element has a shaft extending in the same longitudinal direction as the traction means and having a tensioning device, which is extending transversely thereto and rotatably mounted thereon and into which a proximal end of the traction means is suspendable.

9. The device according to claim 5, wherein the kink in the proximal end section of the control wire can be produced by means of a tool which has two tool parts performing an angular movement relative to one another.

10. The device according to claim 9, wherein the tool parts are designed as two legs connected to one another via a hinge, the control wire being fixable to one of the legs.

11. The device according to claim 10, wherein the legs have grooves aligned with each other and for receiving the control wire and in that on at least one of the legs a clamp for fixing the control wire is provided.

12. A method for converting a device arranged in a working channel of a medical endoscope for use for lithotripsy and removal of a stone captured by a catch basket, being performable after endoscopic retrograde cholangiopancreatography, wherein the device comprises a tube, a control wire guided therein, a catch basket connected distally to the control wire for receiving a stone and a control element engaging the tube and the control wire, and wherein the catch basket is expanded or contracted in a loop-like manner by an axial displacement of the control wire by means of the control element, comprising the following method steps:
Cutting of the control wire in its proximal section, in which it is guided in a guide shaft of the control element;
Kinking of the control wire in a proximal end section to form a hook-shaped configuration;
Suspension of the proximal hook-shaped end section of the control wire in an eyelet of a pulling means of an actuating element used for lithotripsy;
Movement of the hook-shaped end of the control wire and the eyelet of the pulling means in the longitudinal direction into a spiral hose made of metal by tensioning the actuating element, and
further tensioning of the actuating element to generate a force on the catch basket for crushing the stone picked up by the latter.

13. The method according to claim 12, wherein, after the end section provided with the kink has been hooked in, it is first pulled manually in the proximal direction until the eyelet is in contact with the distal end of the spiral hose, in that the proximal end of the pulling means is connected to a tensioning device arranged on the actuating element and the pulling means is then tensioned via the tensioning device, which is designed as a winding spool, until the spiral hose is pushed over the eyelet, in that the tensile force transmitted to the control wire is then increased by further actuation of the tensioning device until the catch basket is moved to the distal end of the tube, and in that the stone picked up by the catch basket is shattered by a further increase in the tensile force.
